(19) United States
(12) Patent Application Publication
(10) Pub. No.: US 2017/0020602 A1
(43) Pub. Date: Jan. 26, 2017

(54) ELECTROSURGICAL INSTRUMENT AND METHOD FOR INSERTING AN APPLICATOR INTO BODY LUMINA

(71) Applicant: OLYMPUS WINTER & IBE GMBH, Hamburg (DE)

(72) Inventors: German KLINK, Berlin (DE); Dana WILLFROTH, Berlin (DE)

(73) Assignee: OLYMPUS WINTER & IBE GMBH, Hamburg (DE)

(21) Appl. No.: 15/124,926

(22) PCT Filed: Mar. 31, 2015
(86) PCT No.: PCT/EP2015/057101

§ 371 Date: Sep. 9, 2016

(30) Foreign Application Priority Data
Apr. 10, 2014 (DE) 10 2014 206 976.9

(51) Int. Cl. A61B 18/14; A61B 18/16

(52) U.S. Cl. CPC A61B 18/1492; A61B 18/1477; A61B 18/1485; A61B 18/16; A61B 2018/00291

(57) ABSTRACT

An electrosurgical instrument, a guiding catheter and a method for inserting an applicator into body lumina along a direction of insertion. The electrosurgical instrument includes a guiding catheter for inserting an applicator into body lumina along a direction of insertion and a counter sleeve, wherein the counter sleeve and the guiding catheter are arranged axially displaceably in relation to one another, and the guiding catheter in a distal region has at least one radial suction opening and the electrosurgical instrument is designed to produce a negative pressure in the guiding catheter, and wherein, furthermore, a cutting element is arranged on the suction opening and/or a distal end of the counter sleeve.

The invention relates to an electrosurgical instrument, in particular for bronchoscopy, comprising a guide catheter for inserting an applicator into a body lumen along an insertion direction.

The invention furthermore relates to a guide catheter and to a method for introducing an applicator into a body lumen.

Electrosurgical instruments and guide catheters of the type mentioned in the introduction are known in the prior art. They are used, in particular, in order to insert therein one or more applicators into a body lumen, particularly into the bronchi.

Electrosurgical applicators may also be referred to as applicator or application probes, and are used in particular for coagulation and/or ablation of biological tissue and/or of deposits. An applicator is generally guided along an insertion direction, and usually movably relative to the guide catheter, in a lumen of the guide catheter. Here, an insertion direction is intended to mean a direction in which a guide catheter or an electrosurgical instrument is inserted into a body lumen. Because of anatomical conditions and since a guide catheter or an electrosurgical instrument can be configured to be flexible, the insertion direction is not necessarily a straight line. For this reason, the forward feed direction, in which the guide catheter or the electrosurgical instrument is essentially pushed forward, is regarded as an insertion direction. The insertion direction therefore also corresponds essentially to a longitudinal axis of the guide catheter or of the electrosurgical instrument.

The applicator usually has an elongate applicator shaft on which a distal applicator electrode, which can form a distal end of the applicator shaft and furthermore a surface component of the applicator shaft, is usually arranged. An applicator tip, which may be configured to be rounded or sharp or which may also be provided with an additional cutting electrode, is usually formed on a distal end of the distal applicator electrode.

Such applicators are used, in particular, to deliver radiofrequency electric current to surrounding tissue, and are also referred to as coagulation or ablation instruments. Monopolar applicators require only one electrode. During use, this electrode interacts with a return or neutral electrode having a large surface, which is likewise connected to the body of a patient. For a bipolar application, applicators with at least two applicator electrodes, preferably a distal and a proximal applicator electrode, are provided. Such bipolar coagulation or ablation instruments may have a radiofrequency (RF) voltage of differing potential (bipolar) applied to them, so that tissue surrounding the electrodes is heated to such an extent that it is dried and/or body proteins are denatured.

Fields of use of electrosurgical instruments are, in particular, bronchoscopy or the treatment of bronchial carcinomas, during which a guide catheter is inserted to a working position or in front of a working position in the body lumen in order to bring the applicator to or in front of a working position. The working position usually lies in a body tissue adjacent to the body lumen. In bronchoscopy, or during the treatment of bronchial carcinomas, for example, this working position lies in the bronchi of the lungs. In order to reach the working position with a distal applicator tip, it is therefore usually necessary to penetrate into the body tissue adjacent to the body lumen. Particularly when the working position lies in the bronchi, to this end the applicator tip often needs to penetrate into a bronchial wall, which may be cartilaginous. For mechanical puncture, applicators therefore often have a mechanically cutting/puncturing applicator tip. This, however, may be disadvantageous since, for example, unintended damage to a guide catheter and/or unintended injuries of body tissue may occur because of such a mechanically cutting/puncturing applicator tip. Furthermore, sometimes high puncturing forces or impacts are necessary in order to allow a mechanically cutting/puncturing applicator tip to penetrate into a cartilaginous bronchial wall, which may lead to an unintended position change of the applicator. Electrosurgical instruments are known in which a cutting electrode with a much smaller surface is provided on the distal end, in addition to the distal coagulation or ablation electrode, as described for example in DE 10 2012 220 682.5. Such electrosurgical instruments, however, can be expensive to produce. Furthermore, for example, it is known from DE 10 2013 212 448.1 to provide an anchoring mechanism with which the distal end of a guide catheter can be fastened on a bronchial wall. From DE 10 2013 212 521.6, it is known to provide a catheter electrode which can be used as a cutting electrode in order to deviate or fix the distal end of the catheter. From DE 10 2013 216 030.5, furthermore, a guide catheter is known which has a deviating mechanism and a stabilizing mechanism, which is used in order to support the distal end of the guide catheter on a bronchial wall. These existing electrosurgical instruments and guide catheters offer various advantages. Other alternatives, and possibly also improved or simplified electrosurgical instruments and guide catheters, are nevertheless desirable.

It is therefore an object of the present invention to provide an electrosurgical instrument, a guide catheter and a method for inserting an applicator into a body lumen, making it easier to bring an applicator to the working position. In particular, it is an object of the present invention to provide an electrosurgical instrument, a guide catheter and a method which facilitate and/or improve the penetration of an applicator tip into a bronchial wall.

This object is achieved according to the invention by an electrosurgical instrument, comprising a guide catheter for inserting an applicator into a body lumen along an insertion direction and a counter sleeve, wherein the counter sleeve and the guide catheter are arranged axially displaceably with respect to one another, and the guide catheter has at least one radial suction opening in a distal region, and the electrosurgical instrument is configured in order to generate a reduced pressure in the guide catheter, and wherein a cutting element is furthermore arranged at the suction opening and/or at a distal end of the counter sleeve.

Besides the guide catheter for inserting an applicator into a body lumen along an insertion direction, the electrosurgical instrument also provides a counter sleeve. The counter sleeve and the guide catheter can be displaced longitudinally axially relative to one another, so that the counter sleeve can be pushed forward or pushed or drawn back in relation to the guide catheter along the insertion direction and/or the guide catheter can be pushed forward or pushed or drawn back in relation to counter sleeve in the insertion direction.

The suction opening in the distal region of the guide catheter is oriented radially, that is to say it is directed radially outward in relation to a longitudinal axis of the guide catheter. The radial suction opening may also be referred to as a lateral suction opening in the side surface of the guide catheter. The electrosurgical instrument is configured in order to generate a reduced pressure in the guide catheter, in particular a temporary reduced pressure, which may in particular be used in order to suction a wall of a body lumen through the suction opening. In this way, the guide catheter can be fixed on the wall of a body lumen. The reduced pressure may for example be generated by a fluid pump, in particular a gas pump, which is preferably arranged at a proximal end of the electrosurgical instrument. Here, a reduced pressure is intended in particular to mean a pressure which is lower than the ambient pressure. When a wall of a body lumen, in particular a bronchial wall, is suctioned through the suction opening of the guide catheter, a part of the wall of the body lumen is generally suctioned into or onto the suction opening.

The electrosurgical instrument furthermore has a cutting element, which is arranged on the suction opening or on a distal end of the counter sleeve. It is also respectively possible to arrange a cutting element at the suction opening and at the distal end of the counter sleeve. When explanations, configurations or advantages relating to a cutting element are indicated below, these also apply correspondingly for two or more cutting elements.

If the cutting element is arranged at the suction opening, the counter sleeve preferably offers a counter surface so that cutting or separation of body tissue takes place between the cutting element at the suction opening and the counter sleeve. If the cutting element is formed at the distal end of the counter sleeve, the edge of the suction opening, or the side surface of the guide catheter, preferably forms a corresponding counter surface so that tissue can be separated or cut between the cutting element and an edge of the suction opening in the guide catheter. If one cutting element is arranged at the suction opening and another cutting element is arranged at the distal end of the counter sleeve, these two cutting elements preferably cooperate in order to cut or separate issue.

The cutting element makes it possible to cut into, or cut off, the wall of a body lumen and thus weaken the wall of the body lumen, in particular a bronchial wall. At the position of the cut, which constitutes a weakening of the wall of the body lumen, an applicator can penetrate more easily into the wall of the body lumen. In particular, the puncture of the bronchial wall by the applicator tip can be facilitated significantly in this way. Further measures which facilitate puncture of the bronchial wall, for example additional cutting electrodes or mechanical tips or blades at the applicator tip, can therefore be omitted, or they can be at least reduced. In combination with the suction opening and the generation of a reduced pressure in the guide catheter, measures for fixing the guide catheter in the region of the working position may furthermore be omitted, or they can be at least reduced.

The invention furthermore has the advantage that no exertion of force, or significantly less exertion of force, is necessary in order to insert the applicator tip into a bronchial wall, and in this case for example flexible applicators in particular can also be used. Existing solutions, conversely, have the disadvantage inter alia that flexible applicators must also have a sufficiently high stiffness so that the bronchial wall can be punctured or broken through without the applicator becoming bent. Furthermore, in solutions in the prior art in which the bronchial wall is punctured without prior cutting-in of the bronchial wall by the applicator tip, it is necessary for the bronchi to have a sufficiently large diameter for a corresponding curvature of the applicator in order to break through and exert a sufficient force for puncture. Furthermore, applicators having a mechanical tip may possibly damage the guide catheter or a working channel of the electrosurgical instrument. The solution according to the invention, conversely, avoids or reduces these disadvantages since, because of the cutting-in of the bronchial wall, it is possible to use a rounded applicator tip and significantly less puncture forces, or even no puncture forces, need to be exerted thereon. Since the suction opening is arranged radially in the guide catheter, use in bronchi with a small diameter is also possible.

According to a preferred configuration of the invention, the cutting element is arranged and configured in order, in the event of an axial relative movement between the counter sleeve and the guide catheter, to separate a part, suctioned through the radial suction opening, of a wall of a body lumen, in particular of a bronchial wall.

In this preferred configuration, the cutting element is arranged and configured in such a way that the cutting of a part, suctioned through the suction opening, of a wall of a body lumen takes place by an axial movement of the guide catheter and/or of the counter sleeve in or counter to the insertion direction, in particular by an axial relative movement between the guide catheter and the counter sleeve. In this way, use even in bronchi with a small diameter is likewise facilitated.

In particular, it is preferred that the suction opening and the distal end of the counter sleeve are arranged and configured in such a way that, in the event of an axial relative movement between the counter sleeve and the guide catheter, a sliding movement takes place between the cutting element and the distal end of the counter sleeve or the edge of the suction opening/side surface of the guide catheter. If a cutting element is respectively provided at the suction opening and at the distal end of the counter sleeve, the suction opening and the distal end of the counter sleeve are preferably arranged and configured in such a way that, in the event of an axial relative movement between the counter sleeve and the guide catheter, a sliding movement takes place between the two cutting elements. The separation or cutting of tissue, in particular the part, suctioned through the radial suction opening, of a wall of a body lumen, in particular of a bronchial wall, is preferably carried out by the sliding movement between the cutting element and a corresponding counter surface at the distal end of the counter sleeve, or at the edge of the suction opening/side surface of the guide catheter, or between two cutting elements. The sliding movement preferably takes place as an axial relative movement or longitudinally axial displacement between the counter sleeve and the guide catheter.

It is particularly preferred that the part of the electrosurgical instrument at which the cutting element is located is moved for the cutting.

If the guide catheter remains in position after the suctioning of a part of the wall of the body lumen, and only a counter sleeve having a cutting element is moved axially as an alternative to the guide catheter, this furthermore has the advantage that the fixing of the guide catheter at the working position is maintained and the guide catheter does not have to be newly positioned or repositioned in order to bring the applicator to the working position.

The sliding movement is preferably carried out by a relative movement between the side surfaces of the guide catheter and the counter sleeve, in particular between the inner side surface of the externally lying element and the outer side surface of the internally lying element. Preferably, sliding contact, or at least sliding past, between the guide catheter and the counter sleeve in immediate proximity takes place at least in the region of the cutting element. Preferably, a sliding movement takes place at least between the suction opening, or the region of the guide catheter side surface enclosing the suction opening, and the distal end of the counter sleeve.

The cutting element may, for example, be configured as a mechanical cutting blade, in order to mechanically cut or separate a part of the wall of the body lumen.

As an alternative or in addition, the cutting element may be configured as an electrosurgical cutting blade, in order to electrosurgically cut or separate the tissue of the wall of the body lumen. An electrosurgical cutting blade is preferably arranged in the form an electrode which is formed with a neutral electrode having a large surface, or with a second electrode, for example on the applicator, on the counter sleeve or on the guide catheter, which is electrically insulated from the cutting blade electrode.

A particularly preferred configuration is obtained when the guide catheter is arranged inside the counter sleeve, the guide catheter preferably being arranged so that it can be extracted from a distal end of the counter sleeve in the insertion direction. In this configuration, the counter sleeve encloses the guide catheter, the inner diameter of the counter sleeve being greater than the outer diameter of the guide catheter. Preferably, the inner diameter of the counter sleeve is only slightly greater than the outer diameter of the guide catheter, so that contact or at least a close sliding movement between the suction opening and the counter sleeve can be produced during an axial relative movement. Yet since, during the suction of the suction opening onto the wall of a body lumen, at least one distal section of the guide catheter bears tightly on the wall of the body lumen, even in the case of a counter sleeve having a larger inner diameter the counter sleeve will slide tightly past the suction opening during a axial relative movement, at least in the region of the suction opening, and the guide catheter is then arranged non-coaxially in the counter sleeve at least in the proximity of the suction opening.

The counter sleeve may preferably be configured as a collection sleeve of the electrosurgical instrument. In particular when the cutting element is formed at the radial suction opening of the guide catheter, a collection sleeve which is preferably present anyway on the electrosurgical instrument may be used as a counter sleeve. In this case, an additional element of the electrosurgical instrument may be omitted. The collection sleeve may, for example, be the working channel of an electrosurgical instrument, in particular of a bronchoscope.

Furthermore, it may be preferred for the counter sleeve to be configured as a separate additional cutting sleeve, which is preferably arranged between the guide catheter and a collection sleeve of the electrosurgical instrument. Such a cutting sleeve may for example have a blunt distal end, and be used as a counter surface for a cutting element formed at the radial suction opening of the guide catheter, or it may itself have a cutting element, preferably at the distal end. The guide catheter is preferably arranged radially inside the cutting sleeve, and the cutting sleeve furthermore is preferably in turn arranged radially inside a collection sleeve of the electrosurgical instrument.

The cutting sleeve may however, for example, also be arranged radially inside the guide catheter, particularly in the inner lumen of a guide catheter. Furthermore, it may be preferred for the distal end of the cutting sleeve to be chamfered, and configured in particular as a cutting element. In this way, the part, suctioned into a radial suction opening of a guide catheter surrounding the cutting sleeve, of a bronchial wall can be advantageously cut into or separated. Another preferred configuration is obtained when the side surface of the cutting sleeve is incised in a radial region and is shaped in such a way that a part of the side surface of the cutting sleeve projects radially inward into the lumen of the cutting sleeve and the side surface of the cutting sleeve therefore has a radial opening. An applicator guided inside the cutting sleeve can emerge from this cutting sleeve through this radial opening. In this case, the part of the side surface projecting into the interior of the cutting sleeve may advantageously form a guide aid, for instance in the form of a ramp, for the applicator emerging through the radial opening.

According to another preferred configuration of the electrosurgical instrument, the cutting element is arranged at a distal edge section of the suction opening, the cutting blade preferably being oriented toward the suction opening. In this configuration, the cutting element is in particular arranged and configured in such a way that the wall of the body lumen can be cut by drawing the guide catheter back counter to the insertion direction.

According to another preferred configuration, the cutting element is arranged at a radially outer edge section of the distal end of the counter sleeve, the cutting blade preferably being oriented toward the distal end of the counter sleeve. This configuration is particularly preferred in order to cut the part, suctioned through the suction opening of the guide catheter, of the wall of the body lumen by pushing the counter sleeve forward against the guide catheter in the insertion direction.

According to another aspect of the invention, the object mentioned in the introduction is achieved by a guide catheter for inserting an applicator into a body lumen along an insertion direction for an above-described electrosurgical instrument, having at least one radial suction opening in a distal region, a cutting element being arranged at the suction opening.

Furthermore, according to another aspect of the invention, the object mentioned in the introduction is achieved by a method for inserting an applicator into a body lumen along an insertion direction, comprising the steps: inserting an above-described electrosurgical instrument into a body lumen in the proximity of a working position, suctioning a wall of the body lumen at the suction opening by generating a reduced pressure in the guide catheter, cutting a part, suctioned at the suction opening, of the wall of the body lumen by generating an axial relative movement between the counter sleeve and the guide catheter, the method furthermore preferably comprising the step: applying a radiofrequency alternating voltage to the cutting element, configured as an electrosurgical cutting blade, and a further electrode is electrically insulated from the electrosurgical cutting blade.

The guide catheter according to the invention and its possible refinements, as well as the method according to the invention and its possible refinements, have features or method steps which in particular make them suitable to be used for an electrosurgical instrument according to the invention and its refinements.

Regarding the advantages, alternative embodiments and embodiment details of these further aspects of the invention and their refinements, reference is made to the preceding description relating to the corresponding features of the electrosurgical instrument.

Preferred embodiments of the invention will be described by way of example with the aid of the appended figures, in which:

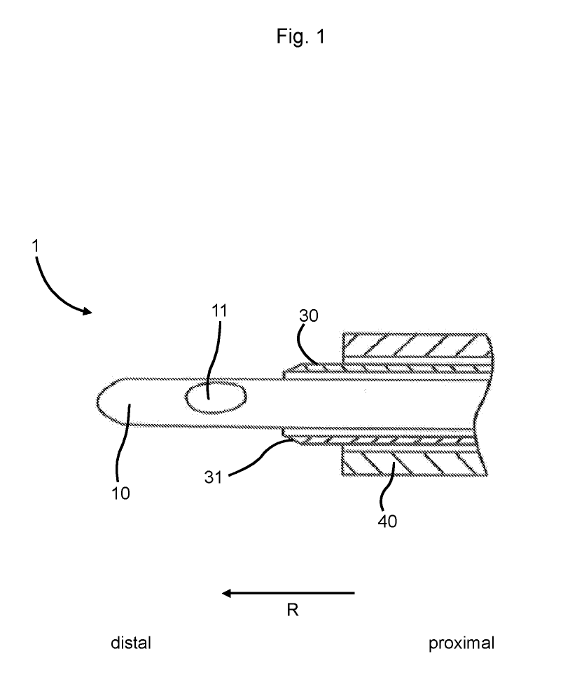

FIG. 1: shows a distal section of a first exemplary embodiment of an electrosurgical instrument according to the invention

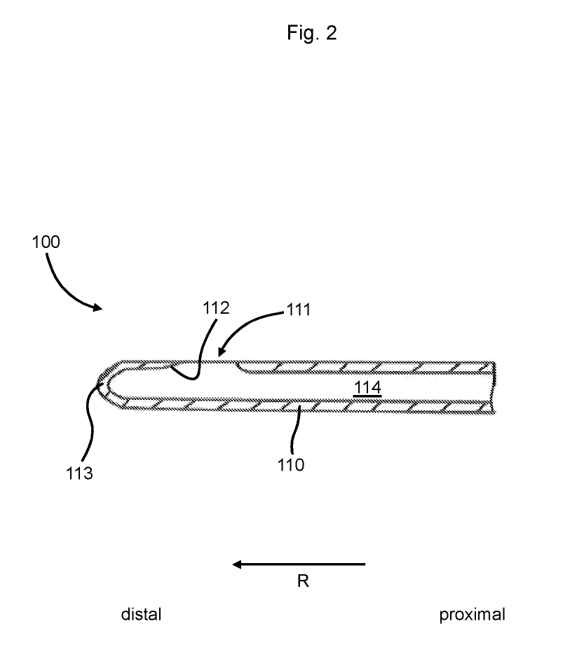

FIG. 2: shows a distal section of an exemplary embodiment of a guide catheter according to the invention;

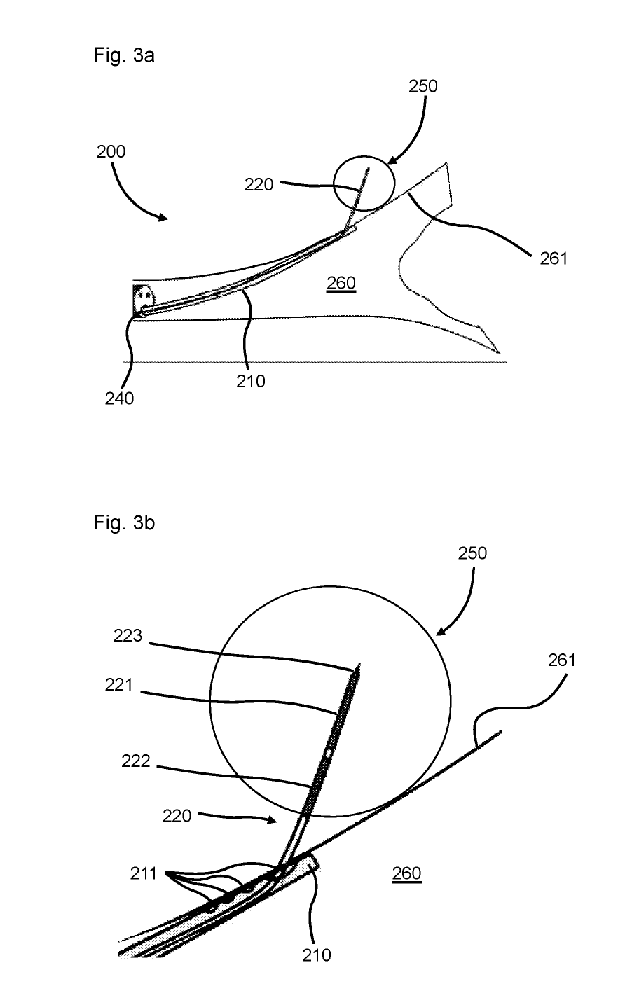

FIG. 3a: shows another exemplary embodiment of an electrosurgical instrument according to the invention at the working position in the bronchi;

FIG. 3b: shows an enlargement of a detail of FIG. 3a; and

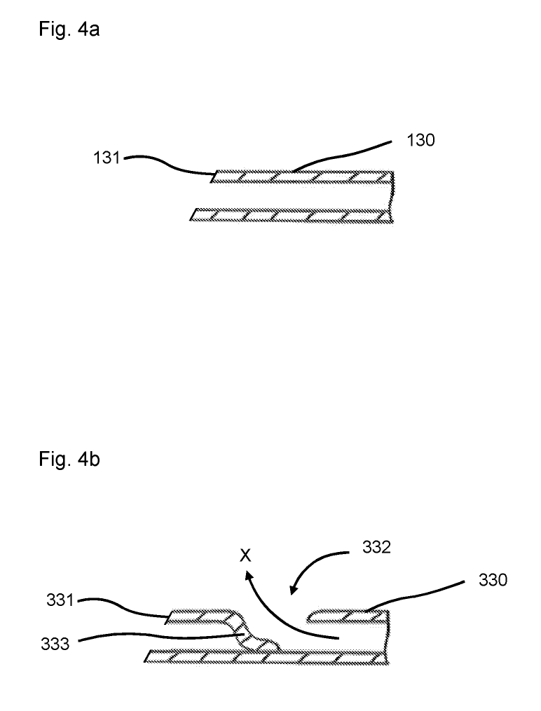

FIGS. 4a,b: show further exemplary embodiments of a counter sleeve configured as a cutting sleeve.

FIG. 1 represents an electrosurgical instrument 1, in particular for bronchoscopy, having a guide catheter 10, a counter sleeve 30 configured as a cutting sleeve and a collection sleeve 40. In its distal region, the guide catheter 10 has a radial suction opening 11. When a reduced pressure is generated in the guide catheter 10, a part of a wall of a body lumen, in particular a part of a bronchial wall, can be suctioned through the suction opening 11.

In the embodiment represented in FIG. 1, the guide catheter 10 is arranged so that it can be displaced axially with respect to the counter or cutting sleeve 30 radially inward in the counter or cutting sleeve 30, so that the counter or cutting sleeve 30 encloses the guide catheter 10 radially outward. The counter or cutting sleeve 30 in turn is likewise arranged so that it can be displaced axially relative to the collection sleeve 40, radially inside the latter, so that the collection sleeve 40 encloses the counter or cutting sleeve 30. The collection sleeve 40 may for example be the working channel of an electrosurgical instrument, in particular of a bronchoscope.

When the distal end of the electrosurgical instrument 1 is inserted into a body lumen in an insertion direction R, the suction opening 11 of the guide catheter 10 is placed in the proximity of the working position, for example in the proximity of a tumor, on a wall of the body lumen and a reduced pressure is set up in the guide catheter 10 so that a part of the wall of the body lumen is suctioned through the suction opening 11. In this way, the guide catheter 10 is fixed at the working position with the suction opening 11.

In order to make it easier for an applicator (not represented in FIG. 1) to penetrate the wall of the body lumen, which is generally a cartilaginous bronchial wall, a mechanical blade 31 is formed on the counter sleeve 30 configured as a cutting sleeve, this blade being arranged at a radially outer edge section of the distal end of the counter sleeve 30 and being oriented toward the distal end of the counter sleeve 30. By pushing the counter or cutting sleeve 30 forward relative to the guide catheter 10 in an insertion direction R, the bronchial wall tissue suctioned through the suction opening 11 can be cut into or cut off. After retraction of the counter or cutting sleeve 30 counter to the insertion direction R in the proximal direction, the bronchial wall part suctioned through the suction opening 11 is then cut and therefore significantly weakened. An applicator introduced to the working position through the guide catheter 10 can therefore emerge from the guide catheter 10 through the suction opening 11 and, at the weakened position of the bronchial wall, penetrate more easily into the latter. Because the guide catheter 10 does not need to be moved in order to cut the bronchial wall in this variant, the suction opening 11 remains at the position intended for the puncture, which makes it possible to insert the applicator at the weakened position of the bronchial wall in the proximity of the working position without newly positioning or repositioning of the guide catheter 10.

FIG. 2 represents an exemplary embodiment of a guide catheter 100, which may preferably also be used in an electrosurgical instrument 1 as shown in FIG. 1. The guide catheter 100 as represented in FIG. 2 has an essentially cylindrical side surface 110 with a rounded distal tip 113 which, during insertion of the guide catheter 100 in an insertion direction R, is used in particular in order to damage neither the working channel of an electrosurgical instrument nor the body lumen into which the guide catheter 100 is inserted.

In the side surface 110 of the guide catheter 100, a lumen 114 is formed through which an applicator (not represented in FIG. 2) can be guided in the guide catheter 100. Furthermore, a reduced pressure can be set up in the lumen 114 of the guide catheter 100 by a corresponding liquid or gas pump (not represented in FIG. 2), in particular a corresponding vacuum pump, at a proximal end of the electrosurgical instrument. A radial suction opening 111 is formed in the side surface 110 of the guide catheter 100. Through this radial suction opening 111, an applicator guided in the lumen 114 of the guide catheter 100 can emerge with its distal end in order to be brought to a working position, for example a tumor. Furthermore, a part of a wall of a body lumen can be suctioned through the radial suction opening 111 when a reduced pressure, i.e. a pressure lower than the ambient pressure, is built up in the lumen 114 of the guide catheter 100. The guide catheter 100 has a cutting element 112, which is arranged at a distal edge section of the suction opening 111, the cutting blade of the cutting element 112 being oriented toward the suction opening 111. In this way, particularly during retraction of the guide catheter 100 counter to the insertion direction R in the proximal direction, a part, suctioned through the suction opening 111, of a wall of a body lumen can be cut into or cut off, so that a distal end of an applicator can penetrate more easily into the bronchial wall at this weakened position. If the guide catheter 100 is moved in order to cut the bronchial wall, before emergence of the applicator tip through the suction opening 111, it is necessary to ensure that the guide catheter 100 with the suction opening 111 is again newly positioned or repositioned over the now weakened position of the bronchial wall in the proximity of the working position.

The guide catheter 100 represented in FIG. 2 may be used in an electrosurgical instrument 1 as represented in FIG. 1, and may cooperate therein with a counter sleeve 30 configured as a cutting sleeve. The guide catheter 100 represented in FIG. 2 may, however, also be used in an electrosurgical instrument in which the cutting sleeve represented in FIG. 1 is not present, but only a collection sleeve since the guide catheter 100 already has a cutting element 112.

FIGS. 3a, 3b represent the use of an electrosurgical instrument 200. The electrosurgical instrument 200 may be configured like the electrosurgical instrument 1 represented in FIG. 1, and/or it may have a guide catheter like the guide catheter 100 represented in FIG. 2.

The electrosurgical instrument 200 has been introduced into a bronchus 260 as a body lumen in the proximity of a working position 250, for example a tumor. The working position 250 lies behind a bronchial wall 261, so that an applicator 220 must penetrate into the bronchial wall 261 in order to reach the working position 250. The applicator 220 has two electrodes 221, 222 electrically insulated from one another, the distal electrode 221 having an electrode tip 223 which facilitates penetration of the applicator 220 into the bronchial wall 261.

The guide catheter 210 is fed out from the distal end of a collection sleeve 240, the applicator 220 in turn being fed out through a suction opening 211 arranged in the distal region of the guide catheter 210. The guide catheter 210 represented in FIG. 3 has a plurality of radial suction openings 211 in the distal region. When a reduced pressure is generated in the guide catheter 210, a part of the bronchial wall 261 is respectively suctioned through the radial suction openings 211. By a cutting element (not represented in FIGS. 3a, 3b) at at least one of the radial suction openings 211 and/or by a cutting element (not represented in FIGS. 3a, 3b) at a cutting sleeve not represented in FIGS. 3a, 3b, the bronchial wall 261 has been weakened by incision or cutting off, so that insertion of the applicator 220 through the bronchial wall 261 to the working position 250 has been facilitated. In particular, the provision of a plurality of radial suction openings 211 improves the fixing of the guide catheter 210 in the proximity of the working position 250.

FIGS. 4a and 4b represent further exemplary embodiments of a counter sleeve 130, 330 configured as a cutting sleeve. The counter sleeve 130, 330 may preferably be arranged radially inside a guide catheter, i.e. in the inner lumen of a guide catheter. The distal end of the counter sleeve 130, 330 is chamfered and is configured as a cutting element 131, 331, and can therefore advantageously cut into or separate a part, suctioned into a radial suction opening of a guide catheter enclosing the counter sleeve 130, 330, of a bronchial wall.

The embodiment of the counter sleeve 330 as represented in FIG. 4b has a further preferred feature: the side surface of the counter sleeve 330 is cut into in a radial region and is shaped in such a way that a part 333 of the side surface of the counter sleeve 330 projects radially inward into the counter sleeve, and the side surface of the counter sleeve 330 has a radial opening 332 through which an applicator guided inside the counter sleeve 330 can emerge from the counter sleeve 330 in the emergence direction X, the part 333 of the side surface projecting into the interior of the counter sleeve 330 advantageously forming a guide aid for the applicator emerging through the radial opening 332.

A feature common to the represented embodiments of the electrosurgical instrument or guide catheter according to the invention is that, by providing at least one cutting element, the part, suctioned through a radial suction opening, of the bronchial wall is weakened by a cut and the penetration of an applicator into the bronchial wall at this weakened position is therefore simplified significantly. The cutting element is in this case arranged and configured in such a way that cutting can be carried out by an essentially axial movement of the guide catheter and/or of the counter sleeve.

### LIST OF REFERENCES

1, 200 electrosurgical instrument
10, 100, 210 guide catheter
11, 111, 211 suction opening
30, 130, 330 counter sleeve
31, 112, 131, 331 cutting element
40, 240 collection sleeve
110 side surface
113 distal tip
114 lumen
220 applicator
221, 222 electrode
223 electrode tip
250 working position
260 bronchus
261 bronchial wall
332 opening
333 part of the side surface of the counter sleeve
R insertion direction
X emergence direction

1. An electrosurgical instrument comprising
a guide catheter for inserting an applicator into a body lumen along an insertion direction and a counter sleeve,
wherein the counter sleeve and the guide catheter are arranged axially displaceably with respect to one another, and
the guide catheter has at least one radial suction opening in a distal region, and the electrosurgical instrument is configured in order to generate a reduced pressure in the guide catheter,
and wherein a cutting element is furthermore arranged at the suction opening and/or at a distal end of the counter sleeve.

2. The electrosurgical instrument as claimed in claim 1,
wherein the cutting element is arranged and configured in order, in the event of an axial relative movement between the counter sleeve and the guide catheter, to cut off a part, suctioned through the radial suction opening, of a wall of a body lumen, in particular of a bronchial wall.

3. The electrosurgical instrument as claimed in claim 1,
wherein the suction opening and the distal end of the counter sleeve are arranged and configured in such a way that, in the event of an axial relative movement between the counter sleeve and the guide catheter, a sliding movement takes place between the cutting element and the distal end of the counter sleeve, or the suction opening.

4. The electrosurgical instrument as claimed in claim 1,
wherein the cutting element is configured as a mechanical cutting blade.

5. The electrosurgical instrument as claimed in claim 1,
wherein the cutting element is configured as an electrosurgical cutting blade.

6. The electrosurgical instrument claimed in claim 1,
wherein the guide catheter is arranged inside the counter sleeve, the guide catheter being arranged so that it can be led out from a distal end of the counter sleeve in the insertion direction.

7. The electrosurgical instrument as claimed in claim 1,
wherein the counter sleeve is arranged inside the guide catheter, the distal end of the counter sleeve being chamfered.

8. The electrosurgical instrument as claimed in claim 1,
wherein the counter sleeve is configured as a collection sleeve of the electrosurgical instrument.

9. The electrosurgical instrument as claimed in claim 1,
wherein the counter sleeve is configured as a separate additional cutting sleeve, which is formed between the guide catheter and a collection sleeve of the electrosurgical instrument.

10. The electrosurgical instrument as claimed in claim 1,
wherein the cutting element is arranged at a distal edge section of the suction opening, the cutting blade being oriented toward the suction opening.

11. The electrosurgical instrument as claimed in claim 1,
wherein the cutting element is arranged at a radially outer edge section of the distal end of the counter sleeve, the cutting blade being oriented toward the distal end of the counter sleeve.

12. A guide catheter for inserting an applicator into a body lumen along an insertion direction for an electrosurgical instrument as claimed in claim 1, having at least one radial suction opening in a distal region, a cutting element being arranged at the suction opening.

13. A method for inserting an applicator into a body lumen along an insertion direction,
comprising the steps:
inserting an electrosurgical instrument as claimed in claim 1 into a body lumen in the proximity of a working position,
suctioning a wall of the body lumen at the suction opening by generating a reduced pressure in the guide catheter,
cutting a part, suctioned at the suction opening, of the wall of the body lumen by generating an axial relative movement between the counter sleeve and the guide catheter.

14. The method as claimed in the claim 13,
wherein:
applying a radiofrequency alternating voltage to the cutting element configured as an electrosurgical cutting blade, and a further electrode, which is electrically insulated from the electrosurgical cutting blade.
